(19) United States
(12) Patent Application Publication
(10) Pub. No.: US 2006/0235388 A1
(43) Pub. Date: Oct. 19, 2006

(54) Pedicular tunneling for decompression and support

(75) Inventors: Jeff R. Justis, Collierville, TN (US); Marc Peterman, Memphis, TN (US); Courtney S. Williams, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/107,426

(22) Filed: Apr. 15, 2005

(51) Int. Cl. A61B 17/70; A61F 2/44

(52) U.S. Cl. 606/61; 623/17.13

(57) ABSTRACT

A system for augmenting a spinal joint comprises a first flexible stabilizing device adapted to extend through a pedicle of a first vertebra, at least a portion of a vertebral body of the first vertebra, at least a portion of a vertebral body of a second vertebra, and a pedicle of the second vertebra.

### BACKGROUND

Severe back pain and nerve damage may be caused by injured, degraded, or diseased spinal joints and particularly, spinal discs. Current methods of treating these damaged spinal discs may include vertebral fusion, nucleus replacements, or motion preservation disc prostheses. Increasingly, facet deterioration and destabilization have also become implicated as a source of pain and neurological complications. Thus, solutions are needed to restore, stabilize, or augment the function of the spinal discs and facets.

### SUMMARY

In on embodiment of the present disclosure, a system for augmenting a spinal joint comprises a first flexible stabilizing device adapted to extend through a pedicle of a first vertebra, at least a portion of a vertebral body of the first vertebra, at least a portion of a vertebral body of a second vertebra, and a pedicle of the second vertebra.

In another embodiment of the present disclosure, a method for augmenting a spinal joint comprises creating a first passage through a pedicle of a first vertebra and into a vertebral body of the first vertebra. The method further comprises creating a second passage through a pedicle of a second vertebra and into a vertebral body of the second vertebra and inserting an elastic stabilizing device through the first and second passages.

In another embodiment of the present disclosure, a system for augmenting a vertebral joint comprises a first means for providing separation to a pair of adjacent articular processes. Said first means extends through at least portions of a first vertebral body, a first pedicle, a second vertebral body, and a second pedicle. The system further comprises a second means for anchoring said first means to said first and second pedicle and a third means for attachment to said first means and for providing compressible support to said first means.

### BRIEF DESCRIPTION OF THE DRAWINGS

FIG. 1 is a left side view of a vertebral column.

FIG. 2 is a partial cross sectional view of an augmentation system according to one embodiment of the present disclosure, implanted within the vertebral column.

FIG. 3 is a partial cross sectional view of an augmentation system according to another embodiment of the present disclosure, implanted within the vertebral column.

FIG. 4 is a partial cross sectional view of an augmentation system according to another embodiment of the present disclosure, implanted within the vertebral column.

### DETAILED DESCRIPTION

The present disclosure relates generally to the field of orthopedic surgery, and more particularly to systems and methods for stabilizing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

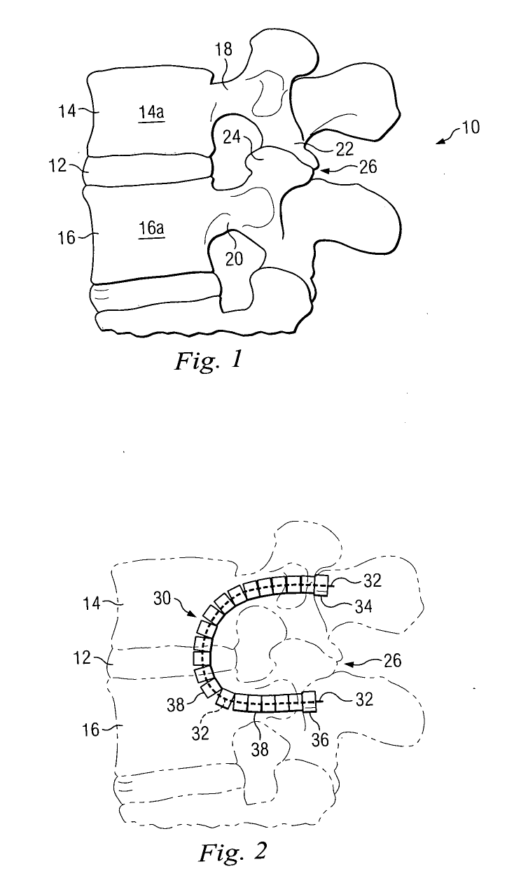

Referring first to FIG. 1, the numeral 10 refers to a vertebral joint which, in this example, includes an injured, diseased, or otherwise damaged intervertebral disc 12 extending between vertebrae 14, 16. The vertebra 14 includes a pedicle 18, and the vertebra 16 includes a pedicle 20. The vertebrae 14, 16 also include vertebral bodies 14a, 16a, respectively. Although the illustration of FIG. 1 depicts the vertebral joint 10 as a lumbar vertebral joint, it is understood that the devices, systems, and methods of this disclosure may also be applied to all regions of the vertebral column, including the cervical and thoracic regions. Furthermore, the devices, systems, and methods of this disclosure may be used in non-spinal orthopedic applications.

The vertebrae 14 further comprises an articular process 22 extending from the pedicle 18 and an articular process 24 extending from the pedicle 20. A facet joint 26 is formed, in part, by the adjacent articular processes 22, 24. Facet joints may also be termed zygapophyseal joints. A healthy facet joint includes a facet capsule extending between the adjacent articular processes. The facet capsule comprises cartilage and synovial fluid to permit the articulating surfaces of the articular processes to remain lubricated and glide over one another. The type of motion permitted by the facet joints is dependent on the region of the vertebral column. For example, in a healthy lumbar region, the facet joints limit rotational motion but permit greater freedom for flexion, extension, and lateral bending motions. By contrast, in a healthy cervical region of the vertebral column, the facet joints permit rotational motion as well as flexion, extension, and lateral bending motions. As the facet joint deteriorates, the facet capsule may become worn, losing its ability to provide a smooth, lubricated interface between the articular surfaces of the articular processes. This may cause pain and limit motion at the affected joint. Removal of an afflicted articular process may result in abnormal motions and loading on the remaining components of the joint. The embodiments described below may be used to decompress a deteriorated facet joint and/or restore more natural motion constraint to a resected joint.

Referring now to FIG. 2, in one embodiment, an augmentation system 30 may be provided to the joint 10 to augment the function of the facet joint 26. The system 30 may comprise a flexible stabilization device 32 which in this embodiment is a bendable wire. In alternative embodiments, the stabilization device may be formed of another type of material that may be bent or flexed into an elastically deformable spring shape. The system 30 may further comprise anchoring devices 34, 36 for connecting the stabilization device at the pedicles 18, 20, respectively, or to other structures of the vertebrae 14, 16, respectively. In this embodiment, the anchoring devices 34, 36 are caps that connect to opposite ends of the stabilization device 32 to prevent migration of the stabilization device and to anchor the stabilization device to the pedicles. In an alternative embodiment, the anchoring devices may be cannulated pedicle screws.

The system 30 may further comprise a series of support members 38 which may extend over the stabilization device 32 to provide compressive support to the implanted stabilization device. The support members 38 may be discrete beads threadable onto the stabilization device 32. The beads may be formed of a compressible material including biocompatible polymeric materials such as silicone, polyurethane, or hydrogels. In an alternative embodiment, the discrete support members may be omitted altogether or may be replaced by a continuous or scored, compressible support sleeve which is attached to the portion of the stabilization device within the vertebral bodies and the intervertebral disc space.

A surgical procedure to implant the augmentation system 30 may be ultra minimally invasive. Using a posterior approach, a small puncture incision may be created in the patient's skin, and an opening may be created in pedicle 18. A passage may be formed from the opening through the pedicle 18 and into the vertebral body 14a. The passage may curve or angle to extend through the bottom endplate of the vertebral body 14a and into the intervertebral disc space between vertebrae 14 and 16. Using either the same puncture incision or a second puncture incision, an opening may be created in pedicle 20. A passage may be formed from this opening through the pedicle 20 and into the vertebral body 16a. The passage may extend through the top endplate of the vertebral body 16a and into the intervertebral disc space to connect with the passage through the vertebral body 14. The resulting passage may be generally C-shaped.

The passages through the vertebral bodies 14a, 16a may be formed using various means for drilling a curved or angled passage in bone, for example, a minimally invasive, flexible, steerable drill may be used. The drill may be steered under radiographic guidance. In alternative embodiments, the passage may be created using a flexible stylet or another type of cutting or tunneling instrument. All or portions of the passage may even be formed by the stabilization device itself. In still another alternative embodiment, a single passage may be formed through both vertebrae 14, 16 using a single approach through either the pedicle 18 or 20.

Referring again to FIG. 2, the stabilization device 32 may be inserted through the passage in pedicle 18, through the vertebral body 14a, through the intervertebral disc space, through the vertebral body 16a, and out through the passage in pedicle 20. As the stabilization device 32 is inserted, it deflects to conform to the C-shape of the passage. The support members 38 may be threaded onto the stabilization device 32 to provide compressive support to the system 30. The anchoring device 34 is connected to an end portion of the stabilization device 32 at the pedicle 18, and the anchoring device 36 is connected to the opposite end portion of the stabilization device 32 at the pedicle 20. The stabilization device 32 may be placed into tension by tightening the anchoring devices 34, 36. As the device 32 is tensioned and the anchoring devices 34, 36 tightened, the support members 38 may become compressed. The support members 38 may serve as support and protection for the stabilization device 32 within its bony passage.

The implanted and anchored stabilization device 32 may provide a dynamic spring action restoring force that draws the articular processes 22, 24 apart, limiting bone to bone contact of the processes and/or relieving pressure on the capsule within the facet joint 26. Static balance may be achieved, and a rigid construct created. Spinal stenosis or other abnormalities impacted by deteriorated facet joints may also be relieved. In embodiments where the facet joint 26 has been removed, the system 30 may restore more natural spacing to the area of the resection and may also restore rotational restraint between the vertebrae 14, 16. It is understood that a second stabilization system may be implanted on the laterally opposite side of the vertebrae 14, 16 from pedicles 18, 20. This second system may be configured and implanted similar to system 30 and therefore will not be described in detail.

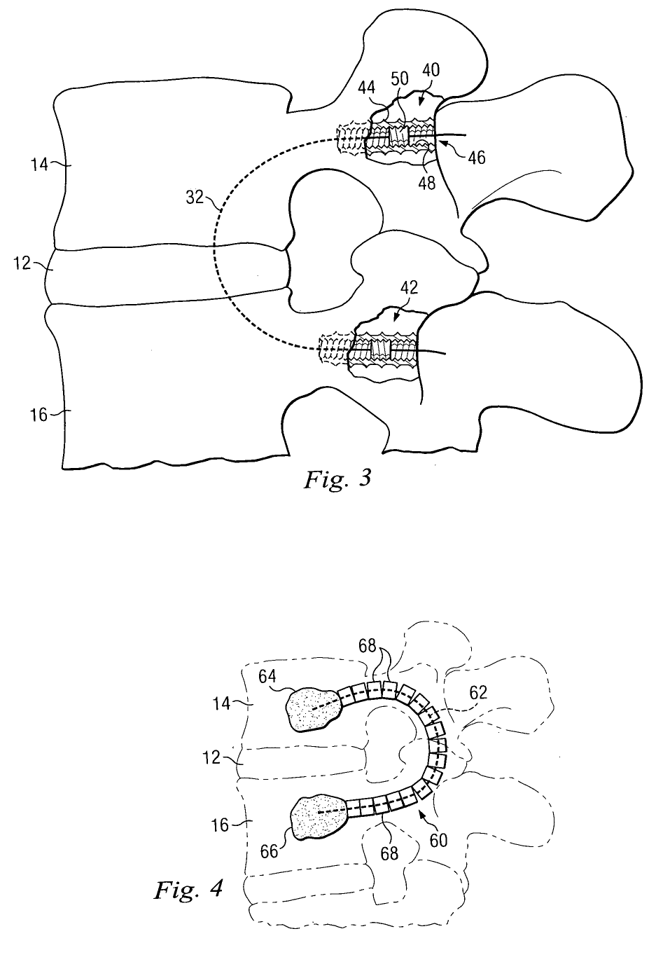

Referring now to FIG. 3, in an alternative embodiment, the stabilization device 32 is anchored to vertebrae 14, 16 by anchoring devices 40, 42, respectively. In this embodiment, the anchoring devices 40, 42 include cannulated pedicle screws. The screw 40 comprises outer threads 44, for fastening to the interior of pedicle 18, and an interior bore 46 for receiving an end portion of the stabilization device 32. The interior bore 46 may comprise threads 48 for engaging an insert 50. Anchoring device 42 may be similarly configured and therefore will not be described in detail.

As described above for the implantation of system 30, a passage is created through the pedicles 18, 20 and the vertebral bodies 14a, 16a. In this embodiment, the anchoring device 40 is threaded into the passage in pedicle 1,8 and the anchoring device 42 is threaded into the passage in pedicle 20. The stabilization device 32 may be inserted through the anchoring device 40, through the vertebral bodies 14a, 16a, and through the anchoring device 42. One end portion of the stabilization device 32 may be fixed to the stabilization device 42 and the opposite end portion may be fixed to the insert 50. The insert 50 may be engaged with the inner threads 48. To tension the stabilization device 42, the insert 50 may be moved along the inner threads 48.

It is understood that a variety of means can be used to create tension in the stabilization device. For example, the insert may be omitted and the stabilization device may be directly tensioned by the anchoring devices. In still other alternative embodiments, solid pedicle screws, suture anchors, knots or other anchoring devices may be used to anchor the stabilization device.

Referring now to FIG. 4, in this embodiment, an augmentation system 60 may be provided to the joint 10 to augment the function of the facet joint 26 and/or the disc 12. The system 60 may comprise a flexible stabilization device 62 which in this embodiment is a bendable wire. In alternative embodiments, the stabilization device may be formed of another type of material that may be bent into an elastically deformable spring shape. The system 60 may further comprise anchoring devices 64, 66 for anchoring end portions of the stabilization device 62 within the vertebral bodies 14a, 16a, respectively. The anchoring devices 64, 66 may be boluses of hardenable material such as bone cement. Acceptable bone cements may include polymethylmethacrylate (PMMA) or calcium phosphate based compounds.

The system 60 may further comprise a series of support members 68 which may extend over the stabilization device 62 to provide compressive support to the implanted stabilization device. The support members 68 may be discrete beads threadable onto the stabilization device 62. The beads may be formed of a compressible material include biocompatible polymeric materials such as silicone, polyurethane, or hydrogels.

A surgical procedure to implant the augmentation system 60 may use an anterior surgical approach. An opening may be created in an anterior wall of the vertebral body 14a and a passage may be created through the vertebral body 14a and through the pedicle 18. An opening may also be created in an anterior wall of the vertebral body 16a and a passage may be created through the vertebral body 16a and through the pedicle 20.

The passages through the vertebral bodies 14a, 16a may be formed using various means for drilling a curved or angled passage in bone, for example, a minimally invasive, flexible, steerable drill may be used. The drill may be steered under radiographic guidance. A central portion of the vertebral bodies 14a, 16a may be compacted or resected to form a void for receiving the anchoring devices 64, 66.

In alternative embodiments, the passage may be created using a flexible stylet or another type of cutting or tunneling instrument. All or portions of the passage may even be formed by the stabilizing device itself. In still another alternative embodiment, the passages through both vertebrae 14, 16 may be formed through a single anterior opening by using a tunneling device capable of creating a C-shaped path.

The stabilization device 62 may be inserted through the opening in vertebral body 14a, through the pedicle 18, through the facet joint 26, through the pedicle 20, and into the vertebral body 16a. As the stabilization device 62 is inserted, it deflects to conform to the C-shape of the passage. The support members 68 may be threaded onto the stabilization device 62 to provide compressive support to the system 60.

The hardenable material for forming the anchoring device 64 may be injected into the vertebral body 14a either anteriorly through the opening in vertebral body 14a or posteriorly through the passage in pedicle 18. Likewise, the hardenable material for forming anchoring device 66 may be injected into the vertebral body 16a. Before the hardenable material sets, the stabilization device 62 may be pretensioned so that the anchoring devices 64, 66 maintain the stabilization device 62 in tension. As the device 62 is tightened, the support members 68 may become compressed. The support members 68 may serve as support and protection for the stabilization device 62 within its bony passage.

In an alternative embodiment, the hardenable material for the anchoring devices 64, 66 may be injected or otherwise provided to the vertebral bodies 14a, 16a, respectively, before the stabilization device is inserted. The material may be allowed to set, after which the stabilization device may be inserted through the anchoring devices. The stabilization device may be tensioned and fastened to the anchoring devices with a clamp, anchoring suture, knot or other anchoring fixture. In still another alternative embodiment, the stabilization device may be anchored to the anterior walls of the vertebral bodies 14a, 16a by suture anchors, plugs, or other anchoring devices known in the art.

The implanted and anchored stabilization device 62 may provide a stable connection across the facet joint and maintain the articular processes 22, 24 in a desired predetermined separation, limiting bone to bone contact of the processes and/or relieving pressure on the capsule within the facet joint 26. Likewise, the stabilization device 62 may draw apart the vertebral bodies 14a, 16a to restore a more natural spacing to the intervertebral disc space. Static balance may be achieved, and a rigid construct created. Spinal stenosis or other abnormalities impacted by deteriorated facet joints may also be relieved. Where the facet joint 26 has been removed, the system 60 may restore more natural spacing to the area of the resection and may also restore rotational restraint between the vertebrae 14, 16. It is understood that a second stabilization system may be implanted on the laterally opposite side of the vertebrae 14, 16 from pedicles 18, 20. This second system may be configured and implanted similar to system 60 and therefore will not be described in detail.

It is understood that in addition to augmenting the function of the facet joint, the augmentation systems described above may be used with spinal disc therapies or intervertebral prosthetic devices. For example, all or a portion of the damaged tissue including the nucleus of disc 12 may be excised. The damaged disc 12 may be replaced by an intervertebral disc prosthesis which may be selected from a variety of devices including any of the prostheses which have been described in U.S. Pat. Nos. 5,674,296; 5,865,846; 6,156,067; 6,001,130; 6,740,118 and in U.S. Patent Application Pub. Nos. 2002/0035400; 2002/0128715; and 2003/0135277; 2004/0225366 which are incorporated by reference herein. It is understood that in an alternative embodiment, the prosthesis may be an intervertebral fusion device. In still another embodiment, the prosthesis may be a nucleus replacement device. The augmentation system may be used together with a prosthetic device to maintain a desired intervertebral height and to reduce and distribute the loads on the joint 10.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A system for augmenting a spinal joint comprising:
a first flexible stabilizing device adapted to extend through
a pedicle of a first vertebra,
at least a portion of a vertebral body of the first vertebra,
at least a portion of a vertebral body of a second vertebra, and
a pedicle of the second vertebra.

2. The system of claim 1 wherein the first stabilizing device further extends through an intervertebral disc space between the first and second vertebrae.

3. The system of claim 1 wherein the first stabilizing device further extends through a facet joint between the first and second vertebrae.

4. The system of claim 1 further comprising:
a first anchoring device attached to the first stabilizing device at the pedicle of the first vertebra.

5. The system of claim 4 further comprising:
a second anchoring device attached to the first stabilizing device at the pedicle of the second vertebra.

6. The system of claim 4 wherein the first anchoring device comprises a cannulated pedicle screw.

7. The system of claim 6 wherein the cannulated pedicle screw comprises an internally threaded bore.

8. The system of claim 1 wherein a first portion of the first stabilizing device is anchored within the vertebral body of the first vertebra and a second portion of the first stabilizing device is anchored within the vertebral body of the second vertebra.

9. The system of claim 8 wherein the first and second portions of the first stabilizing device are anchored with a hardenable material.

10. The system of claim 1 wherein the first stabilizing device is elastically deformable.

11. The system of claim 10 wherein the first stabilizing device is a C-shaped wire.

12. The system of claim 1 further comprising:
a plurality of compressible members attached to the first stabilizing device.

13. The system of claim 1 further comprising:
a second stabilizing device adapted to extend through
an opposite pedicle of the first vertebra,
at least a portion of the vertebral body of the first vertebra,
at least a portion of the vertebral body of the second vertebra, and
an opposite pedicle of the second vertebra.

14. The system of claim 1 wherein a facet joint comprises an articular process of the first vertebra and an articular process of the second vertebra and further wherein the first stabilizing device is adapted to dynamically limit contact between the articular processes.

15. A method for augmenting a spinal joint comprising:
creating a first passage through a pedicle of a first vertebra and into a vertebral body of the first vertebra;
creating a second passage through a pedicle of a second vertebra and into a vertebral body of the second vertebra;
inserting an elastic stabilizing device through the first and second passages.

16. The method of claim 15 further comprising:
creating a passage through an intervertebral disc space between the first and second vertebrae.

17. The method of claim 15 wherein the step of creating the first passage comprises drilling the first passage.

18. The method of claim 15 further comprising attaching an anchoring device to the elastic stabilizing device at the pedicle of the first vertebra.

19. The method of claim 18 wherein the step of attaching comprises threading a cannulated pedicle screw into the pedicle of the first vertebra and attaching the cannulated pedicle screw to the elastic stabilizing device.

20. The method of claim 18 further comprising extending the elastic stabilizing device through a facet joint defined between articular processes of the first and second vertebrae.

21. The method of claim 15 further comprising:
attaching a plurality of compressible members to the elastic stabilizing device.

22. A system for augmenting a vertebral joint comprising:
a first means for providing separation to a pair of adjacent articular processes, said first means extending through at least portions of a first vertebral body, a first pedicle, a second vertebral body, and a second pedicle;
a second means for anchoring said first means to said first and second pedicles; and
a third means for attachment to said first means and for providing compressible support to said first means.

23. The system of claim 22 wherein said second means is adapted for threaded engagement with said first and second pedicles.

24. The system of claim 22 wherein said first means is adapted to flexibly deform to extend through at least portions of said first vertebral body, said first pedicle, said second vertebral body, and said second pedicle.

25. The system of claim 22 wherein said third means comprises a biocompatible polymeric material.
